(19) United States
(12) Patent Application Publication
(10) Pub. No.: US 2004/0197413 A1
(43) Pub. Date: Oct. 7, 2004

(54) Spray dry coacervation systems and methods

(75) Inventors: Eric Y. Sheu, Lafayette, CA (US); Yadong Liu, Fremont, CA (US); Edmund J. Niedzinski, Vacaville, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genteric, Inc., Alameda, CA (US)

(21) Appl. No.: 10/806,896

(22) Filed: Mar. 22, 2004

Related U.S. Application Data
(60) Provisional application No. 60/460,267, filed on Apr. 4, 2003.

(51) Int. Cl. A61K 9/14; A61K 9/50

(52) U.S. Cl. 424/489; 264/5

(57) ABSTRACT

Microparticle formation system and methods that advantageously combine aspects of spray drying and coacervation (or complex coacervation) into a single process. A temperature controllable spray head mounted on a movable stage within a spray chamber produces droplets of solution having controlled size and temperature. The solution can include a first set of one or more solvents and a first set of one or more components or active agents such as one or more APIs. A wet sample receiving pan or reservoir at the bottom of the chamber holds a desired receiving solvent or solvents. The droplets traverse the path between the spray head and reservoir and interact with the solvent in the reservoir. As the droplets traverse the path the first solvent dries. However, the system is configurable such that the first solvent of the droplets does not have to fully dry before the droplets reach the reservoir. This provides flexibility to perform a second process using the reservoir solvent(s) to refine the sprayed droplets, for example to produce microparticles having certain properties unmet by other processes or techniques. The first solvent may be miscible with a reservoir solvent such that coacervation occurs resulting in formation of one or more microparticles of the first set of components or active agents (or a subset thereof). Microparticles formed in this manner have a well-defined or controlled size of low polydispersity not previously available using other techniques. The size and other properties of the formed microparticles, such as integrity and stability, are determined, in part, by the concentration of solution in the droplets. Advantageously, the present invention therefore greatly enhances the capability of spray drying by allowing for the control of several process parameters. For example, four primary process parameters are spray head temperature, flow rate, temperature gradient along the path of the sprayed droplet and the solvent(s) in the reservoir. Other parameters such as pressure can be controlled as well. The process allows for droplets of controlled size and concentration to be provided to the reservoir for further processing, such as coacervation.

### CROSS-REFERENCES TO RELATED APPLICATIONS

This Application is a non-provisional of, and claims the benefit of, U.S. Provisional Application Serial No. 60/460,267, filed Apr. 4, 2002, (Atty. docket 020714-001900US) which is hereby incorporated by reference in its entirety.

### BACKGROUND OF THE INVENTION

Satisfactory in vivo transport of therapeutic agents is vital for many disease indications, from small molecule, peptide, polypeptide, proteins to oligonucleotides and deoxyribonucleic acid (DNA). Efficient delivery often relies on delivery systems that carry the active ingredients. For different active pharmaceutical ingredients (APIs), different delivery systems are needed for maximum bioavailability and minimal toxicity. In many cases, modified release pharmacokinetics is desirable. As a result, systems for delivering liposomes, polymer particles, micelles, emulsions, microemulsions, ligand attached complexes, microcapsules, microparticles and nanoparticles are needed because these systems allow better control of the pharmacokinetic profiles.

Spray dry and co-acervation are well known processes for preparing microparticles and/or microcapsules. A spray dry process is an economically viable process, very simple to operate and is easily scalable. However, such a process produces particles having a limited size range (usually greater than 1 micron), has little control in particle morphology and usually produces microparticles only. In addition, the residual solvent in the particle can create toxicity issues or require additional processes to remove the solvent.

Co-acervation is also a simple and economically viable process. Such a process can be used to produce microparticles or microcapsules depending on simple or complex co-acervation. However, such a process has limited control of the particle size range and polydispersity though it has better control of the particle morphology through hardening kinetics.

In view of the above, there is a need for an improved systems and methods for preparing micro- or nano-particles of narrow particle size distribution (i.e., low polydispersity) and controlled properties. The present invention fulfills this and other needs.

### BRIEF SUMMARY OF THE INVENTION

The present invention provides systems and methods for formulating one or more microparticles having a controlled size. In order to take advantage of aspects of both spray dry and coacervation processes for better control of the pharmacokinetics and toxicity profile, the present invention provides novel systems and apparatus that combines these two processes into a one-step process that fulfills the needs of being able to prepare micro- or nano-particles of narrow particle size distribution (i.e., low polydispersity).

Microparticles formed according to aspects of the present invention exhibit low polydispersity as well as controlled integrity (e.g., shape and morphology) and stability (e.g., against aggregation). A microparticle composition according to the present invention may be formulated to contain desired components such as therapeutic agents. In certain embodiments, the present invention provides a spray-dry co-acervation process that allows for microparticle formulation by combining aspects of spray drying and coacervation processes into a single efficient process.

According to the present invention, a microparticle formation system is provided that advantageously combines aspects of spray drying and coacervation (or complex coacervation) into a single process. A temperature controllable spray head mounted on a movable stage within a spray chamber produces droplets of solution having controlled size and temperature. The solution includes, in certain aspects, a first solvent or mixture of solvents and one or more components that may contain active agents such as one or more active pharmaceutical ingredients (APIs). A wet sample receiving pan, e.g., solvent reservoir, at a controllable distance from the spray heat at the bottom of the chamber holds a desired receiving solvent or solvents. The droplets traverse the path between the spray head and reservoir and interact with the solvent in the reservoir. As the droplets traverse the path the first solvent dries. However, in certain aspects, the system is configured such that the first solvent of the droplets does not have to fully dry before the droplets reach the reservoir. This provides flexibility to perform a second process using the reservoir solvent(s) to refine the sprayed droplets, for example to produce microparticles having desired properties. In certain aspects, for example, the first solvent is miscible with a reservoir solvent such that coacervation occurs resulting in formation of microparticles of the components of the droplets. Microparticles formed in this manner have low polydispersity with controllable average size and morphology not previously feasible using other techniques. The controllable size range and other properties of the formed microparticles, such as integrity and stability, are determined, in part, by the concentration of solution in the droplets. Additionally, other than a coacervation process, other interactions can be used to form microparticles in the reservoir. For example, the interaction in the reservoir can be a precipitation process, a solvent diffusion process, a dispersion process, an emulsification process or a hardening process, depending on the solvents and any additives used. Further, additional components such as process components (e.g., components such as dispersants and stabilizers that facilitate the particle formation process) and formulation components (e.g., components such as active agents that attach to or otherwise become part of the microparticle formed) can be included with a solvent in the reservoir. Advantageously, the present invention therefore greatly enhances the capability of spray drying by allowing for the control of several process parameters. For example, four primary process parameters are spray head temperature, flow rate, temperature gradient along the path of the sprayed droplet and the solvent(s) and additives, if any, in the reservoir. The process allows for droplets of controlled size and concentration to be provided to the reservoir for further processing, such as coacervation.

According to one aspect of the present invention, a system for forming microparticles is provided. The system typically includes a solution source containing a solution including a first solvent or mixture of solvents and a first set of one or more components in a first ratio to the solvent(s), a solvent reservoir containing a second solvent or mixture of solvents with or without additives (second solution), and a spray device in fluid communication with the solution source, the spray device having a spray head configured to produce droplets of the solution having substantially the same average size. The system also typically includes a housing for both the spray head and the solvent reservoir, the housing defining a spray chamber. In operation, solution droplets formed by the spray head within the spray chamber traverse the distance between the spray head and the reservoir and interact with the second solution in the reservoir so as to form microparticles of the first set of components (or a subset of the components), wherein at least one property of each microparticle formed is a function of the average size and the first ratio. In certain aspects, for example, the size of a formed microparticle is a function of the average (droplet) size and the first ratio.

According to another aspect of the present invention, a device is provided for use in a spray-dry-coacervation process. The device typically includes a spray head having an air inlet port, a fluid inlet port, an outlet orifice and a substantially conical cavity portion coupling the inlet ports to the outlet orifice. The device also typically includes a temperature control module slideably coupled to each of an air inlet tube and a fluid inlet tube and configured to control the temperature of fluid entering the conical cavity portion, wherein the air inlet and fluid inlet tubes are connected to the air inlet and fluid inlet ports, respectively, wherein the air inlet tube is fluidly coupled to an air pressure source, and wherein the fluid inlet tube is fluidly coupled to a solution source. The device also typically includes a translation stage configured to adjust the relative distance between the spray head and a target reservoir when coupled to a spray chamber housing. In operation, air flow through the air inlet port causes flow of solution from the solution source through the air inlet port at a rate determined by the rate of air flow through the air inlet port, and wherein the size of droplets of the solution formed at the outlet orifice is determined by the temperature of the solution and the rate of flow of the solution into the cavity portion and the size of the outlet orifice.

According to a further aspect of the present invention, a method of forming micro-particles is provided. The method typically includes providing a solution source including a solution comprising a first solvent or mixture of solvents and a first set of one or more components in a first ratio, forming a plurality of droplets of the solution using a spray head in fluid communication with the solution source, and providing a solvent reservoir at a first distance from the spray head. The reservoir contains a second solvent or mixture of solvents with or without additives (second solution). Droplets received by the reservoir interact with the second solution such that microparticles of the first set of components (or a subset of the components) are formed, wherein at least one property of the microparticles is a function of the first ratio.

According yet a further aspect of the present invention, a microparticle is provided that is formed by providing a solution source containing a solution including a first solvent or mixture of solvents and a first set of one or more components in a first ratio to the solvent(s), forming a droplet of the solution using a droplet forming device in fluid communication with the solution source, and providing a solvent reservoir at a first distance from the device, the reservoir containing a second solvent or mixture of solvents with or without additives (second solution), wherein the droplet interacts with the second solution such that a microparticle of the first set of components (or a subset of the components) is formed, wherein at least one property of the microparticle is a function of the first ratio.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 shows a cross-sectional schematic of a spray-dry coacervation system according to an embodiment of the present invention. FIG. 1 also illustrates aspects of a microparticle formation method according to the present invention.

FIG. 2 illustrates a cross-sectional diagram of a spray head according to an embodiment of the present invention.

FIGS. 3a and 3b illustrate views of the spray device including a translation stage and a temperature control mechanism according to an embodiment of the present invention.

FIG. 4 illustrates an external view of a system housing according to an embodiment of the present invention.

### DETAILED DESCRIPTION OF THE INVENTION

### I. SYSTEMS AND METHODS OF MAKING

The present invention provides systems and processes for preparing a microparticle. In certain aspects, the present invention uses novel spray-dry co-acervation systems and processes. The systems and processes of the present are useful for making particles suitable for delivery of therapeutic agents as well as particles suitable for use in paint, ink, toner, powders, as beads for purification columns, and various other technologies.

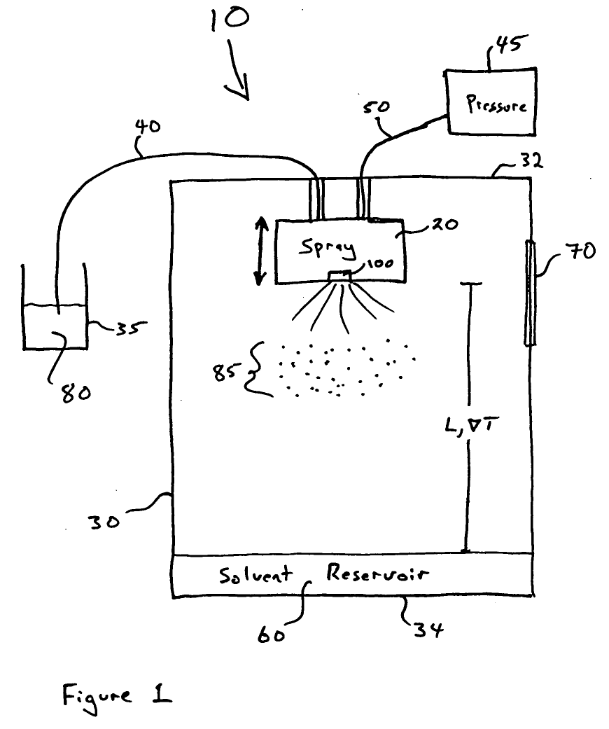

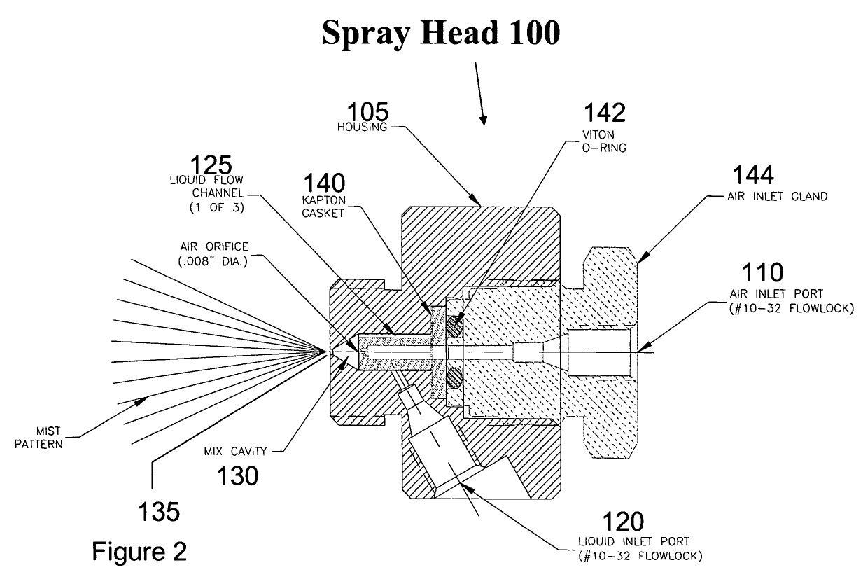

FIG. 1 shows a cross-sectional schematic of a spray-dry coacervation system 10 according to an embodiment of the present invention. FIG. 1 also illustrates aspects of microparticle formation methods according to the present invention. As shown, system 10 according to one embodiment includes a spray device 20 adjustably coupled to top portion 32 of a housing structure 30. Housing structure 30 is preferably configured to hold a solvent reservoir 60 at a bottom portion 34. Spray device 20 includes a spray head 100 configured to produce droplets, e.g., a mist of many droplets, of a solution 80. Spray device 20 is in fluid communication with a solution source 35, e.g., via tubing 40, to supply a solution 80 for droplet formation. Solution source 35 can be a vial, flask or other container configured to hold a solution, such as an aqueous or non-aqueous solution. Spray device 20 is also in fluid communication with pressurized air source 45 via tubing 50. Operation of spray head 100 will be discussed in more detail below with reference to FIG. 2. Briefly, spray head 100 is configured to produce a plurality of droplets 85 of solution 80 having substantially the same size. In preferred aspects, solution 80 is a mixture including a first organic solvent and one or more components or active agents. Preferably, the relative concentration of component or active agent in the solution is predetermined. Solution 80 may also include a mixture of one or more solvents and one or more additives, e.g., components.

Once formed by spray device 20, droplets 85 traverse the distance, L, between spray head 100 and reservoir 60, for example, by gravitational forces and/or pressure forces due to expulsion from spray head 100. Droplets traversing the length L begin to dry, e.g., by evaporation as is well known in conventional spray-dry processes. The rate of drying depends on the size and constituents of solution droplets 85 as well as the time it takes the droplets to traverse distance, L, the pressure in the spray chamber, and the temperature gradient T experienced by the droplets as they traverse L. According to aspects of the present invention, the temperature gradient T experienced by droplets traversing the distance L can be controlled by adjusting one or more parameters, including the distance L, the chamber pressure, the temperature of the environment within spray chamber housing 30 and the temperature of droplets formed by spray device 20. The rate of traversal across distance L can also be, in part, controlled by the rate of flow of droplets formed by spray head 100.

In preferred aspects, the parameters are adjusted so that the solvent in formed droplets 85 does not fully dry before reaching reservoir 60. Thus, in preferred aspects, a plurality of droplets 85, each comprising an organic solvent and a first set of one or more components, e.g., active agents, interact with the solvent reservoir as will be discussed below to produce microparticles all having substantially the same well-defined size. The solvent reservoir may contain a solvent or mixture of solvents with or without additives such as active agents or other components.

FIG. 2 illustrates a cross-sectional diagram of a spray head 100 according to an embodiment of the present invention. As shown, spray head 100 includes a substantially cylindrical housing structure 105, having an optionally varying cross-section as shown, and defining a spray axis 106. Housing 105 is configured with an air inlet port 110 and a fluid inlet port 120. In preferred aspects, air inlet port 110 is coupled to an air pressure source, for example via tubing 50 (FIG. 1) for receiving a pressurized flow of air or other fluid, and fluid inlet port 120 is coupled to solution source 35, for example via tubing 40 (FIG. 1) for receiving solution 80 or other fluid. In preferred aspects, air or fluid flow having a pressure ranging between 0 and 2,000 PSI is used, although other, higher pressures may be used.

In one aspect, housing 105 is also configured with a substantially conical (e.g., angled relative to the axis 106 as shown) mix cavity 130 having an outlet orifice 135. Spray head 100 is configured with one or a plurality, e.g., 3 (one shown), of fluid flow channels 125 coupling the fluid inlet port 120 with the mix cavity 130. Such flow channel(s) are created, for example, by using a gasket 140 configured to provide one or more channels 125 when inserted in a holed-out cylindrical section of housing 105 as shown. Optional parts such as O-ring 142 and gland 144 can be used to allow for simplified machining of housing 105 and more efficient construction of air inlet port 110.

In certain aspects, housing 105 is made of Aluminum or an Aluminum alloy, although other materials may be used as desired. Aluminum is preferred due to ease of machining and manufacture. As an example of construction, a block of aluminum can be machined using well known techniques to produce a hollowed-out housing configuration, such as housing 105 shown in FIG. 2, adapted to receive various tubes, gaskets, O-rings and other inserts as necessary to define the air inlet port 110, fluid inlet port 120, mix chamber 130 and outlet orifice 135 to desired specifications.

In operation, high pressure air flow through inlet port 110 into cavity 130 and out orifice 135 creates a vacuum, or negative pressure at each fluid flow channel 125. The negative pressure at each fluid flow channel 125 operates to induce flow through fluid inlet port 120. That is, the negative pressure operates to draw solution 80 from source 35 through tubing 40 through fluid inlet port 120 through fluid flow channels 125 and into mix cavity 130, where droplets 85 of solution 80 are formed and expelled through orifice 135. The rate of fluid flow from source 35 is generally defined by the negative pressure created, and hence by the pressure of fluid flow generated by pressure source 45. The size of droplets 85 formed is generally defined by the rate of fluid flow from fluid channels 125, by the temperature of fluid in the mix cavity 135, and by the size of outlet orifice 135.

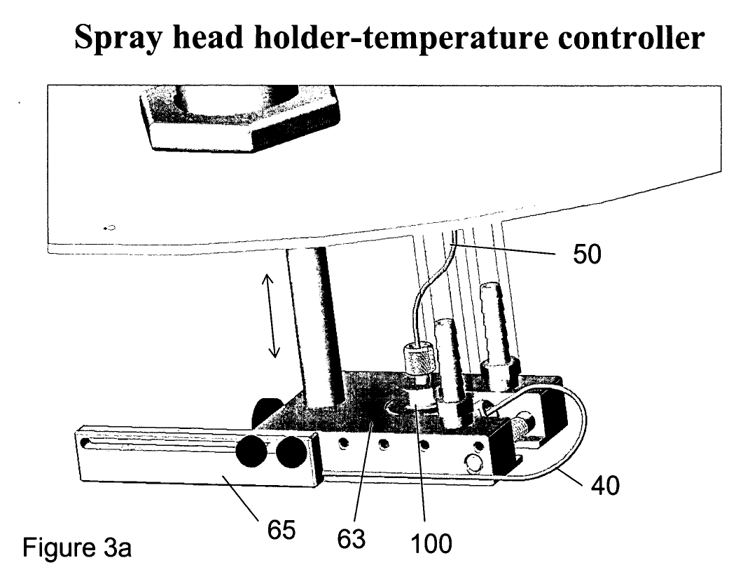

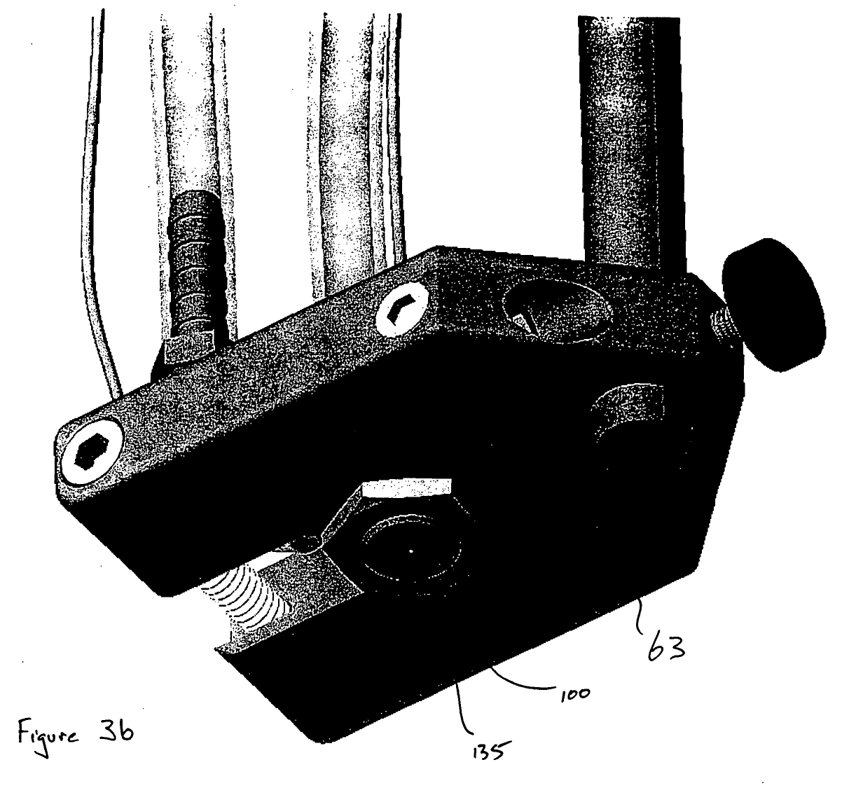

In certain aspects, to control the temperature of fluid in the mix chamber 35, a temperature control mechanism is provided on spray device 20 to control one or both of the temperature of the fluid entering fluid inlet port 120 and the pressurized fluid entering air inlet port 110. In one embodiment as shown in FIGS. 3a and 3b, a temperature control mechanism includes heat conducting metal holder 63 with zero clearance between the holder and the spray head 100. Both flow systems preferably pass through the temperature controlled holder 63 and the spray head 100. Temperature control mechanism also includes, in one embodiment, a metal jacket 65 configured to slidably cover tubing 40 supplying the solution 80 to the inlet port 120. In other embodiments, metal jacket 65 is configured to cover tubing 50 and/or both tubing 40 and 50. Jacket 65 in preferred aspects includes a heat conducting metal. Adjustment of jacket 65 controls the amount of contact of tubing with the jacket and hence provides adjustable temperature control of fluid in the tubing. In certain aspects, a temperature feedback system (not shown) is provided to regulate the temperature to the desired accuracy of a thermocouple (or thermister) specification.

The distance, L, between an output orifice of spray head 100 and the reservoir 60, can be controlled by adjusting the spray head (and holder) upwards or downwards relative to the receiving solvent reservoir as indicated by the double-arrow lines in FIGS. 3a and 3b.

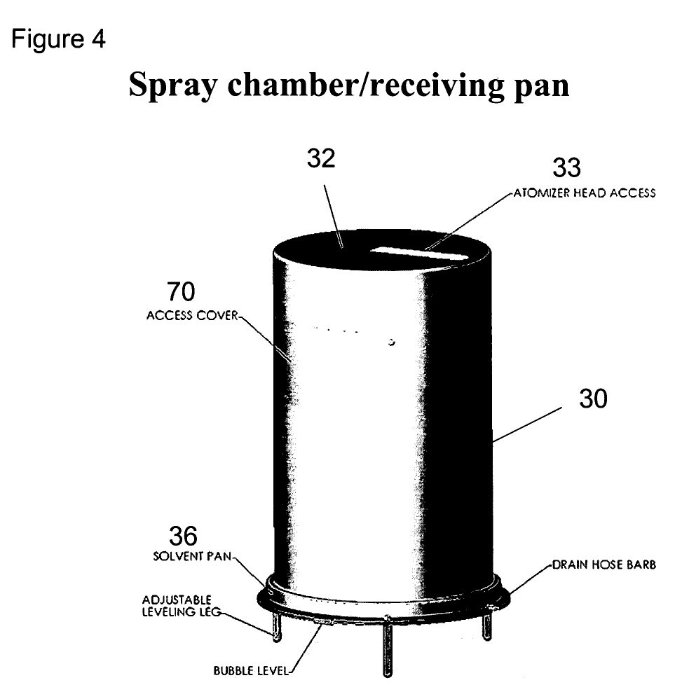

FIG. 4 illustrates an external view of a system housing 30 according to an embodiment of the present invention. As shown housing 30 includes an access panel or door 70 and a spray head access portion 33 in a top portion 32 of the housing. A solvent pan portion 36 is provided for holding solvent(s) and a drain hose 37 is provided for ease of removal of solvent(s) and/or other material, e.g., including microparticles, in pan 36. Adjustable leveling legs and a bubble level are optionally provided to allow for stability of the system.

Referring back to FIG. 1, when droplets 85 reach solvent reservoir 60, the droplets interact with the solvent and other components in reservoir 60. In preferred aspects, the interaction involves a coacervation process. For example, the solvent in the reservoir 60 may be selected to be miscible with the solvent in the solution droplets 85 so as to facilitate coacervation of the droplets and formation of microparticles of the component or components in the solution droplets. The system in other aspects may be configured to facilitate other interaction processes with droplets 85 (either partially dried or in some cases fully dried), such as, for example, precipitation, dispersion, emulsification, solvent diffusion and hardening processes.

The methods of the present invention can be preferably used for making microparticles having well defined sizes. In general, the size of a formed microparticle is a function of the concentration (e.g., ratio of component to solvent) of the solution 80. For example, in a spray-dry coacervation process, a plurality of droplets all having substantially the same average size and substantially the same concentration are formed. These droplets also all traverse substantially the same path (e.g., traversing the same distance L and experiencing the same temperature gradient) before interacting with the reservoir bath. Upon interaction with the reservoir bath, all droplets are substantially self-similar.

For a droplet having a specific size and a specific concentration, the amount of component is defined, and the size of the microparticle is also defined. For example, for a droplet of 5.0% w/v of a first component in a first solvent, and having a size of approximately 1.0 μm, the resulting microparticle will be approximately 50 nm in size as estimated by the equation: droplet size×concentration ratio (% w/v). The present invention can be used to produce droplets ranging in size from the sub-micron range or smaller up to millimeters or greater, and droplet concentration ranging from approximately 60% w/v (or greater) down to 1%, or 0.1% or even 0.01% or smaller, thus providing microparticles ranging in size from 1,000 nm or greater down to 10 nm or smaller.

In certain aspects, additional properties of the microparticles are enhanced. For example, the systems and methods of the present invention also allow for the production of microparticles having enhanced stability and integrity relative to prior methods. Such properties are also typically determined as a function of the solution concentration, droplet size, and the reservoir contents. The integrity of a particle includes properties such as surface morphology, e.g., regular rounded shape, uniformity, and homogeneity, e.g., in concentration and in size. The stability of a particle in certain cases is a function of a particle's uniformity and surface properties such as roughness and hydrophobicity.

In some embodiments, one or more components are added to reservoir 60. Such components can include process components and formulation components. Process components typically include desired components that facilitate the process of microparticle formation. Examples of process components include, stabilizers and dispersants (e.g., to help prevent agglomeration). Formulation components are desired components that are intended as constituents of a microparticle. Examples include active agents. For example, some active agents may not be stable or may be damaged during certain process portions such as during droplet formation or during path traversal due to temperature fluctuation. It may therefore be desirable to include such particle constituents in the solvent reservoir. Additionally, it may be desirable to have certain active agents attach to the first component (from the solution droplets) during particle formation in the reservoir. For example, a component included in the reservoir may be a nucleic acid having an affinity for the first component.

### II. COMPOSITIONS

In one embodiment, the present invention provides a microparticle comprising one or more components or active agents, such as, for example, one or more active pharmaceutical ingredients (APIs).

The systems and methods of the present invention advantageously produce microparticles having low polydispersity. Typical microencapsulation techniques produce particles having heterogeneous size distributions ranging, for example, between 0.01 μm to about 1000 μm, or between 0.1 μm to about 100 μm, or between about 0.5 μm to about 50 μm, depending on the technique used. The present invention advantageously allows for the production of microparticles having controlled sizes in the nanometer range (e.g., between 1 nm to 1,000 nm (1 μm)) or lower and in the micron range (e.g., between 1.0 μm and 1,000 μm) or higher. Furthermore, the present invention advantageously allows for the production of a plurality of microparticles that are substantially monodisperse in size, e.g., having a substantially homogeneous size distribution, e.g., less than approximately 10% polydispersity. By producing microparticles that have well controlled properties, release of an active agent can be better controlled. Thus, the invention permits improvements in the preparation of modified release formulations for therapeutic applications.

The present invention is useful for preparing and manufacturing particles as described in U.S. Provisional Patent Application Serial No. 60/424,882, (Atty. Docket No. 020714-000510US) filed Nov. 8, 2002, and [??] (Atty. Docket No. 020714-000520US), filed Mar. 28, 2003, the contents of which are hereby incorporated by reference in their entirety. The present invention is also useful in preparing particles including formulations, active agents, components, etc., as described in U.S. patent application Ser. No. 60/407,375, filed Aug. 30, 2002 (Atty. Docket 020714-000600US), and [??], filed Mar. 11, 2003 (Atty. Docket 020714-000610US), and U.S. Pat. Nos. 5,837,693, 5,885,971, 6,004,944, 6,225,290, 6255,289, 6,258,789, 5,527,928, 5,744,625, 5,892,071, 5,869,715, 5,824,812, 5,925,623, 6,043,390, 6,372,722 and 6,248,720, the contents of each being hereby incorporated by reference in its entirety.

### III. ACTIVE AGENTS

A wide range of active agents can be employed in the present invention, such as nucleic acid, proteins, small molecules and various other components or agents in whole or in part. Preferably, an active agent is incorporated into a microparticle during formation of the microparticle. In one embodiment, for example, an active agent is incorporated into the solution, e.g., an aqueous or non-aqueous solution, used to form droplets. In other embodiments, an active agent is alternatively or additionally incorporated into the solvent in the solvent reservoir. For example, certain active agents may not react well to stresses or conditions experienced while traversing the path from the spray head to the reservoir, and are thus optimally included in the solvent reservoir for attachment to, or interaction with, components or active agents in the received droplets.

In certain aspects, an active agent is present in a range of about 0.01% to about 60% w/v of the droplet solution. In a preferred aspect, an active agent is present in a range of about 0.01% to about 10% w/v, such as about 0.1% to 1.0% w/v of the solution.

In certain preferred aspects, an active agent is nucleic acid (e.g., viral nucleic acid, or viral particle). The DNA of interest can be any DNA encoding any protein. For example, intravenous protein therapy is appropriate in treating a mammalian subject having an inherited or acquired disease associated with a specific protein deficiency (e.g., diabetes, hemophilia, anemia, severe combined immunodeficiency). Such protein deficient states are amenable to treatment by replacement therapy, i.e., expression of a protein to restore the normal blood stream levels of the protein to at least normal levels. Secretion of a therapeutic protein to the gastrointestinal tract (e.g., by secretion of the protein into the saliva, pancreatic juices, or other mucosal secretion) is appropriate where, for example, the subject suffers from a protein deficiency associated with absorption of nutrients (e.g., deficiency in intrinsic factor) or digestion (e.g., deficiencies in various pancreatic enzymes).

Alternatively, the mammalian subject may have a condition which is amenable to treatment by expression or over-expression of a protein which is either normally present in a healthy mammalian subject or is foreign to the mammalian subject. For example, intravenous protein therapy can be used in treatment of a mammalian subject having a viral (e.g., human immunodeficiency virus (HIV), Epstein-Barr virus (EBV), herpes simplex virus (HSV), bacterial, fungal, and/or parasitic infection, particularly where the infection is chronic, i.e., persisting over a relatively long period of time. The gene therapy of the invention may also be used to enhance expression of a protein present in a normal mammal, or to express a protein not normally present in a normal mammal, in order to achieve a desired effect (e.g., to enhance a normal metabolic process). For example, a cell of a dairy cow may be transformed with DNA encoding bovine growth hormone (BGH) in order to enhance levels of BGH in the blood stream and enhance milk production.

The DNA of interest is preferably obtained from a source of the same species as the mammalian subject to be treated (e.g. human to human), but this is not an absolute requirement. DNA obtained from a species different from the mammalian subject can also be used, particularly where the amino acid sequences of the proteins are highly conserved and the xenogeneic protein is not highly immunogenic so as to elicit a significant, undesirable antibody response against the protein in the mammalian host.

Exemplary, preferred DNAs of interest include DNA encoding insulin, growth hormone, clotting factor VIII, intrinsic factor, and erythropoietin. Of particular interest is intravenous protein therapy of a mammalian subject (e.g., a bovine, canine, feline, equine, or human subject, preferably a bovine or human subject, more preferably a human subject) by expression of DNA encoding a protein (e.g., insulin, growth hormone, clotting factor VIII, or erythropoietin) in a transformed mammalian cell. Preferably, the subject is a human subject and the DNA expressed encodes a human protein (e.g., human insulin, human growth hormone, human clotting factor VIII, or human erythropoietin). Other exemplary DNAs of interest include tissue plasminogen activator (tPA), urokinase, streptokinase, acidic fibroblast growth factor, basic fibroblast growth factor, tumor necrosis factor alpha, tumor necrosis factor β, transforming growth factor β,β-interferon, platelet-derived growth factor, endothelian, and soluble CD4. Table 1 provides a list of exemplary proteins and protein classes which can be delivered using particles formed according to the present invention.

TABLE 1
SPECIFIC EXEMPLARY PROTEINS

| | |
|---|---|
| insulin | interferon-α2B |
| human growth hormone (hGH) | transforming growth factor (TGF) |
| erythropoietin (EPO) | ciliary neurite transforming factor (CNTF) |
| clotting factor VIII | insulin-like growth factor-1 (IGF-1) |
| bovine growth hormone (BGH) | granulocyte macrophage colony stimulating |
| | factor (GM-CSF) |
| platelet derived growth factor (PDGF) | interferon-α2A |
| clotting factor VIII | brain-derived neurite factor (BDNF) |
| thrombopoietin (TPO) | insulintropin |
| IL-1 | tissue plasminogen activator (tPA) |
| IL-2 | urokinase |
| IL-1 RA | streptokinase |
| superoxide dismutase (SOD) | adenosine deamidase |
| catalase | calcitonin |
| fibroblast growth factor (FGF) (acidic or | arginase |
| basic) | |
| neurite growth factor (NGF) | phenylalanine ammonia lyase |
| granulocyte colony stimulating factor (G- | γ-interferon |
| CSF) | β-interferon |
| L-asparaginase | pepsin |
| uricase | trysin |
| chymotrypsin | elastase |
| carboxypeptidase | lactase |
| sucrase | intrinsic factor |
| calcitonin | parathyroid hormone(PTH)-like hormone |
| Ob gene product | cholecystokinin (CCK) |
| glucagon | insulinotrophic hormone |

EXEMPLARY CLASSES OF PROTEINS

| | |
|---|---|
| proteases | pituitary hormones |
| protease inhibitors | growth factors |
| cytokines | somatomedin |
| chemokines | immunoglobulins |
| gonadotrophins | interleukins |
| chemotactins | interferons |
| lipid-binding proteins | |

In certain aspects, therapeutic agents, which may be administered using the present invention, can be any of a variety of drugs, which are selected to be an appropriate treatment for the disease to be treated. Table 2 sets forth various small molecules suitable for use in the present invention.

TABLE 2
Exemplary Drug Classes and Drug

| Class of Therapeutic | Specific Examples |
|---|---|
| antineoplastic agents | vincristine, doxorubicin, mitoxantrone, |
| | camptothecin, cisplatin, bleomycin, |
| | cyclophosphamide, methotrexate, streptozotocin |
| antitumor agents | actinomycin D, vincristine, vinblastine, cystine |
| | arabinoside, anthracyclines, alkylative agents, |
| | platinum compounds, taxol |
| antimetabolites | |
| nucleoside analogs | methotrexate, purine, pyrimidine analogs. |
| anti-infective agents | |
| local anesthetics | dibucaine, chlorpromazine |
| β-adrenergic blockers | propranolol, timolol, labetolol |
| antihypertensive | clonidine, hydralazine |
| agents | |
| anti-depressants | imipramine, amitriptyline, doxepim |
| anti-conversants | phenytoin |
| antihistamines | diphenhydramine, chlorphenirimine, |
| | promethazine |
| antibiotic/antibacterial | gentamycin, ciprofloxacin, cefoxitin |
| agents | |
| antifungal agents | miconazole, terconazole, econazole, isoconazole, |
| | butaconazole, clotrimazole, itraconazole, nystatin, |
| | naftifine, amphotericin B |
| antiparasitic agents | |
| hormones | estrogen, testosterone, androgen, leuprolide |
| hormone antagonists | |
| immunomodulators | |
| neurotransmitter | |
| antagonists | |
| antiglaucoma agents | |
| vitamins | vitamin A, vitamin D, |
| narcotics | morphine, |
| imaging agent | |

### IV. ADMINISTRATION

A microparticle comprising a component or an active agent (e.g., DNA) of interest may be administered by any suitable technique known, including, but not limited to, orally, parenterally, transmucosally (e.g., sublingually or via buccal administration), topically, transdermally, rectally and via inhalation (e.g., nasal or deep lung inhalation). Parenteral administration includes, but is not limited to, intravenous, intraarterial, intraperitoneal, subcutaneous, intramuscular, intrathecal, and intraarticular.

For buccal and/or oral administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycolate), or wetting agents (for example, wetting agents). The tablets can be coated according to methods well known in the art.

The compositions can also be administered retroductally, such as by introduced into the lumen of a salivary gland duct. Suitable methods of retroductal introduction of the composition to the salivary gland duct include, for example, cannulation or injection of the composition into the salivary gland duct using a syringe, cannula, catheter, or shunt. The type of syringe, cannula, catheter, or shunt used is not a critical part of the invention. One of skill in the art will appreciate that multiple types of syringes, cannulas, catheters, or shunts may be used to administer compositions according to the methods of the present invention.

Retroductal delivery of the composition using the methods of the present invention may be via gravity or an assisted delivery system. Suitable assisted delivery systems include metering pumps, controlled-infusion pumps and osmotic pumps. The particular delivery system or device is not a critical aspect of the invention. One of skill in the art will appreciate that multiple types of assisted delivery systems may be used to deliver compositions according to the methods of the present invention. Suitable delivery systems and devices are described in U.S. Pat. Nos. 5,492,534, 5,562,654, 5,637,095, 5,672,167, and 5,755,691. One of skill in the art will also appreciate that the infusion rate for delivery of the composition may be varied. Suitable infusion rates may be from about 0.005 ml/min to about 1 ml/minute, preferably from about 0.01 ml/min to about 0.8 ml/min., more preferably from about 0.025 ml/min. to about 0.6 ml/min. It is particularly preferred that the infusion rate is about 0.05 ml/min.

In one embodiment, when DNA of interest is introduced using a microparticle of the present invention, one first determines in vitro the optimal values for the DNA:microparticle ratios and the absolute concentrations of DNA and lipid as a function of cell death and transformation efficiency for the particular type of cell to be transformed. These values can then be used in or extrapolated for use in vivo transformation. The in vitro determinations of these values can be readily carried out using techniques, which are well known in the art.

Preferably, the DNA construct contains a promoter to facilitate expression of the DNA of interest within a cell, such as a pancreatic cell, or salivary gland cell. Preferably, the promoter is a strong, eukaryotic promoter. Exemplary eukaryotic promoters include promoters from cytomegalovirus (CMV), mouse mammary tumor virus (MMTV), Rous sarcoma virus (RSV), and adenovirus. More specifically, exemplary promoters include the promoter from the immediate early gene of human CMV (Boshart et al., Cell 41:521-530, 1985) and the promoter from the long terminal repeat (LTR) of RSV (Gorman et al., Proc. Natl. Acad. Sci. USA 79:6777-6781, 1982). Of these two promoters, the CMV promoter is preferred as it provides for higher levels of expression than the RSV promoter. The DNA of interest may be inserted into a construct so that the therapeutic protein is expressed as a fusion protein (e.g., a fusion protein having β-galactosidase or a portion thereof at the N-terminus and the therapeutic protein at the C-terminal portion). Production of a fusion protein can facilitate identification of transformed cells expressing the protein (e.g., by enzyme-linked immunosorbent assay (ELISA) using an antibody which binds to the fusion protein).

It may also be desirable to produce altered forms of the therapeutic proteins that are, for example, protease resistant or have enhanced activity relative to the wild-type protein. Further, where the therapeutic protein is a hormone, it may be desirable to alter the protein's ability to form dimers or multimeric complexes. For example, insulin modified so as to prevent its dimerization has a more rapid onset of action relative to wild-type, dimerized insulin.

The construct containing the DNA of interest can also be designed so as to provide for site-specific integration into the genome of the target cell. For example, a construct can be produced such that the DNA of interest and the promoter to which it is operably linked are flanked by the position-specific integration markers of Saccharomyces cerevisiae Ty3. The construct for site-specific integration additionally contains DNA encoding a position-specific endonuclease, which recognizes the integration markers. Such constructs take advantage of the homology between the Ty3 retrotransposon and various animal retroviruses. The Ty3 retrotransposon facilitates insertion of the DNA of interest into the 5′ flanking region of many different tRNA genes, thus providing for more efficient integration of the DNA of interest without adverse effect upon the recombinant cell produced. Methods and compositions for preparation of such site-specific constructs are described in U.S. Pat. No. 5,292,662, incorporated herein by reference with respect to the construction and use of such site-specific insertion vectors.

### V. DEFINITIONS

As used herein, the term “microparticle” or “particle” refers to a composition including one or more components and/or one or more active agents, either in solution or as a solid. A “microparticle” or “particle” may also refer to a composition that can be used to deliver one or more components and/or one or more active agents, either in solution or as a solid, wherein the component is surrounded by an encapsulation material or embedded in various materials. The encapsulation material coats an interior comprising the component or active agent, such as an API. The size of a microparticle generally refers to an average diameter of the microparticle.

As used herein, “encapsulation” can refer to a formulation that provides a compound with full encapsulation, partial encapsulation, or combinations thereof.

The term “nucleic acid” refers to a polymer containing at least two nucleotides. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleotides are the monomeric units of nucleic acid polymers. A “polynucleotide” is distinguished here from an “oligonucleotide” by containing more than 80 monomeric units; oligonucleotides contain from 2 to 80 nucleotides. The term nucleic acid includes deoxyribonucleic acid (DNA) and ribonucleic acid (RNA). The term encompasses sequences that include any of the known base analogs of DNA and RNA.

DNA may be in the form of anti-sense, plasmid DNA, parts of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, ribozymes, chimeric sequences, or derivatives of these groups.

“Antisense” is a polynucleotide that interferes with the function of DNA and/or RNA. This may result in suppression of expression. Natural nucleic acids have a phosphate backbone, artificial nucleic acids may contain other types of backbones and bases. These include PNAs (peptide nucleic acids), phosphothionates, and other variants of the phosphate backbone of native nucleic acids. In addition, DNA and RNA may be single, double, triple, or quadruple stranded.

The term “recombinant DNA molecule” as used herein refers to a DNA molecule that is comprised of segments of DNA joined together by means of molecular biological techniques. “Expression cassette” refers to a natural or recombinantly produced polynucleotide molecule that is capable of expressing protein(s). A DNA expression cassette typically includes a promoter (allowing transcription initiation), and a sequence encoding one or more proteins. Optionally, the expression cassette may include trancriptional enhancers, noncoding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences.

The term “gene” refers to a nucleic acid (e.g., DNA) sequence that comprises coding sequences necessary for the production of a polypeptide or precursor (e.g., myosin heavy chain). The polypeptide can be encoded by a full length coding sequence or by any portion of the coding sequence so long as the desired activity or functional properties (e.g., enzymatic activity, ligand binding, signal transduction, and the like) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ nontranslated sequences. The term “gene” encompasses both cDNA and genomic forms of a gene. A genomic form or clone of a gene contains the coding region interrupted with noncoding sequences termed “introns” or “intervening regions” or “intervening sequences.” Introns are segments of a gene, which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Upregulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the term “aqueous phase” refers to a composition comprising in whole, or in part, water.

The term “lipid” refers to a group of organic compounds that are esters such as fatty acid esters, and are characterized by being insoluble in water but soluble in many organic solvents. They are usually divided in at least three classes: (1) “simple lipids” which include fats and oils as well as waxes; (2) “compound lipids” which include phospholipids and glycblipids; (3) “derived lipids” such as steroids.

The term “amphipathic lipid” refers, in part, to any suitable material wherein the hydrophobic portion of the lipid material orients into a hydrophobic phase, while a hydrophilic portion orients toward the aqueous phase. Amphipathic lipids are usually the major component of a lipid vesicle. Hydrophilic characteristics derive from the presence of polar or charged groups such as carbohydrates, phosphato, carboxylic, sulfato, amino, sulfhydryl, nitro, hydroxy and other like groups. Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Other compounds lacking in phosphorus, such as sphingolipid, glycosphingolipid families, diacylglycerols and β-acyloxyacids, are also within the group designated as amphipathic lipids. Additionally, the amphipathic lipid described above can be mixed with other lipids including triglycerides and sterols.

The term “anionic lipid” refers to any lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, and other anionic modifying groups joined to neutral lipids.

The term “cationic lipid” refers to any of a number of lipid species, which carry a net positive charge at a selective pH, such as physiological pH. Such lipids include, but are not limited to, N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N-(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of cationic lipids are available which can be used in the present invention. These include, for example, LIPOFECTIN® (commercially available cationic liposomes comprising DOTMA and 1,2-dioleoyl-sn-3 -phosphoethanolamine (“DOPE”), from GIBCO/BRL, Grand Island, N.Y., USA); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (“DOSPA”) and(“DOPE”), from GIBCO/BRL); and TRANSFECTAM® (commercially available cationic lipids comprising dioctadecylamidoglycyl carboxyspermine (“DOGS”) in ethanol from Promega Corp., Madison, Wis., USA). The following lipids are cationic and have a positive charge at below physiological pH: DODAP, DODMA, DMDMA and the like.

All Patents, Patent Applications and references included and mentioned herein are hereby incorporated by reference in their entirety.

While the invention has been described by way of example and in terms of the specific embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A method of forming micro-particles, comprising:
providing a solution source containing a solution including a first solvent and a first component in a first ratio;
forming a plurality of droplets of said solution using a spray head in fluid communication with the solution source; and
providing a solvent reservoir at a first distance from the spray head, said reservoir containing a second solvent, wherein droplets received by the reservoir interact with the second solvent such that microparticles of the first component are formed, wherein at least one property of the microparticles is a function of the first ratio.

2. The method of claim 1, wherein the first ratio is a weight-by-volume percentage and wherein the size of a microparticle is substantially equal to the size of a droplet multiplied by the first ratio.

3. The method of claim 2, wherein the first ratio is less than about 5% weight-by-volume of first component to first solvent, wherein the average size of each droplet is less than approximately 1.0 μm, such that the average size of each microparticle is less than about 50 nm.

4. The method of claim 2, wherein the first ratio is less than about 10% weight-by-volume of first component to first solvent, wherein the average size of each droplet is less than approximately 10 μm, such that the average size of each microparticle is less than about 1,000 nm.

5. The method of claim 2, wherein the first ratio is between about 5% to 10% weight-by-volume of first component to first solvent, wherein the average size of each droplet is between about 1.0 μm and 10 μm, such that the average size of each microparticle is between about 50 nm and 1,000 nm.

6. The method of claim 1, wherein the first component includes a nucleic acid.

7. The method of claim 6, wherein the nucleic acid is selected from the group consisting of DNA, RNA, DNA/RNA hybrids, an antisense oligonucleotide, a chimeric DNA-RNA polymer, a ribozyme, and a plasmid DNA.

8. The method of claim 1, wherein the first component includes a one or more active pharmaceutical ingredients (APIs).

9. The method of claim 1, further including adjusting the first distance between the spray head and the reservoir.

10. The method of claim 1, wherein the plurality of droplets all have substantially the same average size based on the flow rate and temperature of the solution entering the spray head, the method further comprising adjusting the average size of the droplets by controlling one or both of the flow rate and temperature of solution entering the spray head.

11. The method of claim 1, further comprising adding a second component to the second solvent in the reservoir.

12. The method of claim 11, wherein the second component includes one of a process component and a formulation component.

13. The method of claim 12, wherein the process component includes one of a dispersant and a stabilizer.

14. The method of claim 11, wherein the second component includes a nucleic acid having an affinity for the first component.

15. The method of claim 1, wherein the second solvent is immiscible with said first solvent.

16. The method of claim 1, wherein the at least one property includes one or more of the size, the integrity and the stability of the microparticle.

17. A system for forming microparticles, comprising:
a solution source containing a solution including a first solvent and a first component in a first ratio;
a solvent reservoir containing a second solvent;
a spray device in fluid communication with said solution source, said spray device having a spray head configured to produce droplets of said solution having substantially the same average size; and
a housing for both the spray head and the solvent reservoir, said housing defining a spray chamber,
wherein solution droplets formed by the spray head within the spray chamber traverse the distance between the spray head and the reservoir and interact with the second solvent in the reservoir so as to form microparticles of the first component, wherein at least one property of each microparticle formed is a function of the average size and the first ratio.

18. The system of claim 17, wherein the distance between the spray head and the reservoir is adjustable.

19. The system of claim 17, wherein the first ratio is a weight-by-volume percentage and wherein the size of a microparticle is substantially equal to the size of a droplet multiplied by the first ratio.

20. The system of claim 19, wherein the first ratio is less than about 5% weight-by-volume of first component to first solvent, wherein the average size of each droplet is less than approximately 1.0 μm, such that the average size of each microparticle is less than about 50 nm.

21. The system of claim 19, wherein the first ratio is less than about 10% weight-by-volume of first component to first solvent, wherein the average size of each droplet is less than approximately 10 μm, such that the average size of each microparticle is less than about 1,000 nm.

22. The system of claim 19, wherein the first ratio is between about 5% to 10% weight-by-volume of first component to first solvent, wherein the average size of each droplet is between about 1.0 μm and 10 μm, such that the average size of each microparticle is between about 50 nm and 1,000 mn.

23. The system of claim 17, wherein the average size of the droplets is a function of the flow rate and temperature of solution entering the spray head, wherein the spray device is configured with a temperature control mechanism and a flow control mechanism.

24. The system of claim 23, wherein the temperature control mechanism includes a slideable metal jacket positioned proximal a solution inlet tube, whereby adjustably sliding the jacket over the tube adjusts the temperature of the solution entering the spray head through the inlet tube.

25. The system of claim 24, wherein the flow control mechanism includes a pressurized air source coupled to an air inlet port on the spray head, wherein the air flow from the air inlet port defines the flow rate of the solution from a solution inlet port on the spray head.

26. The system of claim 17, wherein the distance between the spray head and the reservoir is shorter than a distance in which the first solvent of the droplets would fully dry while traversing.

27. The system of claim 17, wherein the spray device includes a movable stage coupled to the housing, said movable stage allowing for adjustment of the distance between the spray head and the reservoir.

28. The system of claim 17, wherein the first component includes a nucleic acid.

29. The system of claim 28, wherein the nucleic acid is selected from the group consisting of DNA, RNA, DNA/RNA hybrids, an antisense oligonucleotide, a chimeric DNA-RNA polymer, a ribozyme, and a plasmid DNA.

30. The system of claim 17, wherein the first component includes a one or more active pharmaceutical ingredients (APIs).

31. The system of claim 17, wherein the second solvent is immiscible with said first solvent.

32. The system of claim 17, wherein the at least one property includes one or more of the size, the integrity and the stability of the microparticle.

33. The system of claim 17, wherein the solvent reservoir includes a second component with the second solvent, said second component including one of a process component and a formulation component.

34. A device for use in a spray-dry-coacervation process, the device comprising:
a spray head having an air inlet port, a fluid inlet port, an outlet orifice and a substantially conical cavity portion coupling the inlet ports to the outlet orifice;
a temperature control module slideably coupled to each of an air inlet tube and a fluid inlet tube and configured to control the temperature of fluid entering the conical cavity portion, wherein said air inlet and fluid inlet tubes are connected to the air inlet and fluid inlet ports, respectively, wherein the air inlet tube is fluidly coupled to an air pressure source, and wherein the fluid inlet tube is fluidly coupled to a solution source; and
a translation stage configured to adjust the relative distance between the spray head and a target reservoir when coupled to a spray chamber housing,
wherein air flow through the air inlet port causes flow of solution from the solution source through the air inlet port at a rate determined by the rate of air flow through the air inlet port, and wherein the size of droplets of the solution formed at the outlet orifice is determined by the temperature of the solution and the rate of flow of the solution into the cavity portion and the size of the outlet orifice.

35. The device of claim 34, wherein the temperature control module includes a slideable metal jacket positioned proximal the fluid inlet tube, whereby adjustably sliding the jacket over the fluid inlet tube adjusts the temperature of the solution entering the cavity portion through the fluid inlet tube.

36. The device of claim 34, wherein the device is configured to produce droplets of the solution all having substantially the same average size, said average size ranging in diameter from approximately 0.5 μm to approximately 100 μm.

37. The device of claim 34, wherein the solution is an aqueous solution.

38. The system of claim 17, wherein the solution is an aqueous solution.

39. The method of claim 1, wherein the solution is an aqueous solution.

40. A microparticle formed by:
providing a solution source containing a solution including a first solvent and a first component in a first ratio;
forming a droplet of said solution using a droplet forming device in fluid communication with the solution source; and
providing a solvent reservoir at a first distance from the device, said reservoir containing a second solvent, wherein the droplet interacts with the second solvent such that a microparticle of the first component is formed, wherein at least one property of the microparticle is a function of the first ratio.

41. The microparticle of claim 40, wherein the first ratio is a weight-by-volume percentage and wherein the size of the microparticle is substantially equal to the size of the droplet multiplied by the first ratio.

42. The microparticle of claim 41, wherein the first ratio is less than about 5% weight-by-volume of first component to first solvent, wherein the size of the droplet is less than approximately 1.0 μm, such that the size of the microparticle is less than about 50 nm.

43. The microparticle of claim 41, wherein the first ratio is less than about 10% weight-by-volume of first component to first solvent, wherein the size of the droplet is less than approximately 10 μm, such that the size of the microparticle is less than about 1,000 nm.

44. The microparticle of claim 41, wherein the first ratio is between about 5% to 10% weight-by-volume of first component to first solvent, wherein the size of the droplet is between about 1.0 μm and 10 μm, such that the size of the microparticle is between about 50 nm and 1,000 nm.

45. The microparticle of claim 40, wherein the first component includes a nucleic acid.

46. The microparticle of claim 45, wherein the nucleic acid is selected from the group consisting of DNA, RNA, DNA/RNA hybrids, an antisense oligonucleotide, a chimeric DNA-RNA polymer, a ribozyme, and a plasmid DNA.

47. The microparticle of claim 40, wherein the first component includes a one or more active pharmaceutical ingredients (APIs).

48. The microparticle of claim 40, wherein the microparticle is further formed by adding a second component to the second solvent in the reservoir, wherein the second component includes on of a process component and a formulation component.

49. The microparticle of claim 48, wherein the second component is one of a dispersant and a stabilizer.

50. The microparticle of claim 48, wherein the second component includes a nucleic acid having an affinity for the first component, such that the formed microparticle includes the second component.

51. The microparticle of claim 40, wherein the first solvent is immiscible with the second solvent.

52. The microparticle of claim 40, wherein the solution is an aqueous solution.

53. The microparticle of claim 40, wherein the first component includes β-interferon.

54. The system of claim 17, wherein the first component includes β-interferon.

55. The method of claim 1, wherein the first component includes β-interferon.

56. A method of forming micro-particles, comprising:
providing a solution source containing a solution including a first solvent and a set of one or more first components in a first ratio;
forming a plurality of droplets of said solution using a spray head in fluid communication with the solution source; and
providing a solution reservoir at a first distance from the spray head, said reservoir containing a second solvent, wherein droplets received by the reservoir interact with the second solvent such that microparticles of the first component are formed, wherein at least one property of the microparticles is a function of the first ratio.

57. The method of claim 56, wherein said reservoir includes one or more additives.
